(19) United States
(12) Patent Application Publication
(10) Pub. No.: US 2017/0224780 A1
(43) Pub. Date: Aug. 10, 2017

(54) METHODS FOR PREVENTING TRANSPLANTATION-INDUCED FOLLICLE ACTIVATION

(71) Applicant: TEL HASHOMER MEDICAL RESEARCH INFRASTRUCTURE AND SERVICES LTD., Ramat Gan (IL)

(72) Inventors: Dror MEIROW, Jerusalem (IL); Hadassa RONESS, Neve Daniel (IL)

(73) Assignee: TEL HASHOMER MEDICAL RESEARCH INFRASTRUCTURE AND SERVICES LTD., Ramat Gan (IL)

(21) Appl. No.: 15/581,001

(22) Filed: Apr. 28, 2017

Related U.S. Application Data
(63) Continuation-in-part of application No. 15/506,496, filed on Feb. 24, 2017.
(60) Provisional application No. 62/044,259, filed on Aug. 31, 2014.

(51) Int. Cl. A61K 38/22; A61B 17/435; A61K 35/54

(52) U.S. Cl. CPC A61K 38/22; A61K 35/54; A61B 17/435

(57) ABSTRACT

Provided are compositions and methods for preventing premature follicle activation and loss induced by transplantation of ovaries or tissues derived from ovaries, thereby preserving fertility in a subject.

### FIELD OF THE INVENTION

The present invention relates to compositions and methods for preventing premature follicle activation and loss induced by transplantation of ovaries or tissues derived from ovaries, thereby preserving fertility in a subject.

### BACKGROUND OF THE INVENTION

The ovarian primordial follicle pool in humans is established during embryonic development. This pool constitutes the complete supply of oocytes that have the potential to ovulate through life. The population of primordial (non-growing) follicles (‘reserve’) containing diplotene oocytes is arrested in the first meiotic prophase. A ‘reserve’ of primordial follicles is the number of primordial follicles at any given age and is ultimately depleted by continuous recruitment and degeneration until exhausted. After primordial follicle development is initiated, a small number of the follicles are destined to ovulate while the rest undergo atresia. The factors that control the initiation of primordial follicle development are crucial for female fertility.

Ovarian tissue cryopreservation and transplantation (OTCP-TP) has proven to be a successful fertility preservation technique with more than 80 live births reported by various teams across the world and over 90% return of ovarian function in graft recipients (e.g. Meirow et al., Fertil Steril., 106(2):467-474, 2016). However, a significant follicle loss accompanies the OTCP-TP process (Kawamura et al., Human Reproduction, 30(11): 2457-2460, 2015; Silber, Assist Reprod. Genet., 33:1595-1603, 2016).

It was shown by some of the inventors of the present invention that follicle loss associates with accelerated primordial follicle activation (Kalich-Philosoph et al. Sci Transl Med, 5(185):185, 2013; Roness et al., Cell Cycle, 12(20): 3245-3246, 2013).

Anti-mullerian hormone (AMH) is produced by the granulosa cells of early growing follicles. AMH serum levels are currently used as a marker of ovarian follicle reservoir. Studies have shown that AMH participates in selection points of follicle development (Skinner, Hum Reprod Update., 11(5):461-471, 2005). AMH was also shown to have a role in maintaining primordial follicle dormancy under physiological conditions (Reddy et al., Trends in Endoc. & Metabol., 21(2):96-103, 2010). However, studies conducted in vitro (2-day-old mouse ovarian culture) and in AMH-knockout female mice did not examine the effect of exogenous AMH (Durlinger et al., Reproduction, 2002, 124: 601-609), while other studies concluded that exogenous AMH does not affect the number of primordial follicles (Durlinger et al., Endocrinology, 143(3):1076-1084, 2002).

There remains an unmet need for therapeutic approaches using pharmacological agents rather than invasive procedures, for preserving the oocyte pool and preventing undesirable and premature follicle activation and loss induced by ovary transplantation.

### SUMMARY OF THE INVENTION

The present invention provides pharmaceutical compositions, kits and methods for protecting fertility under or following ovary transplantation. Thus, the methods of the invention are useful for preventing premature follicle activation and loss, inhibiting undesired or premature activation of follicles, preserving the depot of primordial follicles, postponing premature menopause, reducing the side effects associated with premature menopause, in women undergoing ovary transplantation. Surprisingly, as exemplified below, rAMH prevented transplantation-induced follicle activation in women undergoing ovarian tissue cryopreservation and transplantation (OTCP-TP). These findings contribute to the optimization and improvement of OTCP-TP.

The term “premature follicle activation” is interchangeable with the term “artificially induced follicle activation” and “induced follicle activation” and refers to accelerated and/or premature follicle activation and follicle loss (also termed ‘follicle burn-out’) which is induced by an acute damage, such as medical treatment, including, but not limited to ovary transplantation among other medical treatments that may induce follicle burn out.

Accordingly, the pharmaceutical compositions, kits and methods disclosed herein provide a therapeutic platform for reducing complications and preserving fertility in women undergoing OTCP-TP.

The terms “transplantation” and “ovarian tissue cryopreservation and transplantation”, or “OTCP-TP” as used herein are interchangeable and include, but are not limited to, OTCP-TP protocols as known to date.

In some embodiments, there is provided a method of inhibiting premature follicle activation comprising administering to a subject in need thereof a therapeutically effective amount of a pharmaceutical composition comprising a compound selected from the group consisting of anti-mullerian hormone, anti-mullerian hormone agonist, and antiMIR of anti-mullerian hormone, wherein the premature follicle activation is induced by ovarian transplantation.

In some embodiments, the compound is anti-mullerian hormone.

In some embodiments, the ovarian transplantation comprises transplantation of ovarian tissue or whole ovary. Each possibility is a separate embodiment of the invention.

In some embodiments, said pharmaceutical composition is administered during any one or more of the following: prior to said transplantation, during said transplantation, and post said transplantation. Each possibility is a separate embodiment of the invention.

In some embodiments, said pharmaceutical composition is administered prior to and during, said transplantation.

In some embodiments, said pharmaceutical composition is administered simultaneously with said transplantation. In some embodiments, said pharmaceutical composition is further administered after said transplantation.

In some embodiments, the method further comprising administering to said subject at least one follicle reserve protective compound.

In some embodiments, said subject is a female subject in her reproductive years.

In some embodiments, said transplantation comprises ovarian tissue cryopreservation and transplantation.

In some embodiments, said transplantation is an autologous transplantation.

In some embodiments, said transplantation is an orthotropic transplantation.

In some embodiments, there is provided a method for ovarian tissue transplantation, the method comprising:
(a) obtaining ovarian cortex from a first subject;
(b) preparing the ovarian cortex, or fragments thereof for transplantation;
(c) preparing an ovarian medulla of a second subject for transplantation;
(d) attaching the prepared ovarian cortex, or fragments thereof, to the prepared ovarian medulla; and
(e) administering a pharmaceutical composition comprising a pharmaceutically effective amount of AMH.

In some embodiments, said first subject and said second subject are the same subject.

In some embodiments, said first subject is a donor and said second subject is a recipient.

In some embodiments, the method further comprising preserving the ovarian cortex, or fragments thereof prior to preparing the ovarian cortex for transplantation.

In some embodiments, preserving comprises freezing.

In some embodiments, preparing comprises thawing.

In some embodiments, the subject is having a disease or disorder requiring treatment, and wherein said obtaining ovarian cortex is performed prior to said treatment. In some embodiments, said treatment is chemotherapy.

In some embodiments, said subject is a healthy woman wishing to preserve her fertility for nonmedical reasons.

In some embodiments, there is provided a pharmaceutical composition comprising a compound selected from the group consisting of anti-mullerian hormone, anti-mullerian hormone agonist, and antiMIR of anti-mullerian hormone, for use as a medicament for the inhibition of premature follicle activation induced by ovary transplantation of ovarian tissue or whole ovary.

In some embodiments, there is provided a pharmaceutical composition comprising a compound selected from the group consisting of anti-mullerian hormone, anti-mullerian hormone agonist, and antiMIR of anti-mullerian hormone, for use in the inhibition of premature follicle activation induced by transplantation of ovarian tissue or whole ovary.

In some embodiments, the compound is anti-mullerian hormone.

In some embodiments, said use occurs during any one or more of the following: prior to said transplantation, during said transplantation, and post said transplantation. Each possibility is a separate embodiment of the invention.

In some embodiments, said use occurs prior to and during said transplantation.

In some embodiments, said use occurs simultaneously with said transplantation. In some embodiments, said use further occurs post transplantation.

In some embodiments, said transplantation comprises ovarian tissue cryopreservation and transplantation.

In some embodiments, said transplantation is an autologous transplantation.

In some embodiments, said transplantation is an orthotropic transplantation.

In some embodiments, there is provided pharmaceutical composition comprising a compound selected from the group consisting of anti-mullerian hormone, anti-mullerian hormone agonist, and antiMIR of anti-mullerian hormone, for use as a medicament in combination with at least one follicle reserve protective compound for the inhibition of premature follicle activation induced by transplantation of ovarian tissue or whole ovary.

In some embodiments, the at least one follicle reserve protective compound comprises sphingosine-1-phosphate, tamoxifen, GnRH, trichloro(dioxoethylene-O,O′) or a combination thereof.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Exemplary embodiments are illustrated in referenced figures. It is intended that the embodiments and figures disclosed herein are to be considered illustrative rather than restrictive. The figures are listed below.

FIG. 1A shows follicle count in grafts of marmoset ovarian cortical tissues untransplanted (control) and 3 or 7 days post transplantation.

FIG. 1B shows follicle count in grafts of bovine ovarian cortical tissues untransplanted (control) and 3 or 7 days post transplantation.

FIG. 1C shows follicle count in grafts of human ovarian cortical tissues untransplanted (control) and 3 or 7 days post transplantation.

FIG. 2A shows GF/PMF ratio in grafts of marmoset ovarian cortical tissues untransplanted (control) and 3 or 7 days post transplantation.

FIG. 2B shows GF/PMF ratio in grafts of bovine ovarian cortical tissues untransplanted (control) and 3 or 7 days post transplantation.

FIG. 2C shows GF/PMF ratio in grafts of human ovarian cortical tissues to untransplanted (control) and 3 or 7 days post transplantation.

FIG. 3A shows immunostained tissue sections of marmoset ovarian cortical tissues untransplanted (control) and 7 days post transplantation (transplant).

FIG. 3B shows immunostained tissue sections of bovine ovarian cortical tissues untransplanted (control) and 7 days post transplantation (transplant).

FIG. 3C shows immunostained tissue sections of human ovarian cortical tissues untransplanted (control) and 7 days post transplantation (transplant).

FIG. 4A shows follicle count in tissue sections of marmoset ovarian cortical tissues untransplanted (black column), transplanted (gray column) and transplanted together with treatment with rAMH (white column).

FIG. 4B shows GF/PMF ratio in grafts of marmoset ovarian cortical tissues untransplanted (black column), transplanted (gray column) and transplanted together with treatment with rAMH (white column).

FIG. 5 shows immunostained tissue sections of marmoset ovarian cortical tissues untransplanted (control), transplanted and transplanted together with treatment with rAMH.

FIG. 6 shows immunofluorescent images of immunostained tissue sections of marmoset ovarian cortical tissues transplanted but not treated with rAMH and transplanted together with treatment with rAMH.

FIG. 7 shows count of primordial (white columns) and growing (black columns) follicles in whole ovaries cultured with medium alone (control; No Rx), medium and 200 ng/ml AMH (AMH), in the presence of phosphoramide mustard (PM) for 4 hours followed by medium alone, and initially cultured with PM together with 200 ng/ml AMH (PM+AMH). **p<0.01 No Rx compared with PM, ***p<0.001 PM+AMH compared with PM alone.

FIG. 8 shows the ratio of growing to dormant (primordial stage) follicles in each of the treatment groups presented in FIG. 7. *p<0.05 No Rx compared with PM, and PM+AMH compared with PM alone.

### DETAILED DESCRIPTION OF THE INVENTION

Studies of fetal, neonatal, and adult human ovaries have shown that several millions of non-growing follicles (NGF) are established at around five months of gestational age. This number declines to the point where approximately 1,000 remain at the onset of menopause, which occurs at an average age of 50-51 years. It was further estimated that for 95% of women by the age of 30 years only 12% of their maximum pre-birth NGF population remains and by the age of 40 years only 3% remains. Although about one million oocytes are present at birth in the human ovary, only about 500 (about 0.05%) of these ovulate, where the rest are wasted.

The terms “non-growing follicles”, “NGF”, “resting follicles” and “dormant follicles” as used herein are interchangeable and refer to a depot of follicles prior to activation, which have the potential to become activated under suitable natural or artificial factors and conditions.

The high incidence of cancer in girls and young women challenges reproductive medicine-cancer is curable in the majority of cases but at the cost of rendering the treated patients sterile. Almost 6% of women of reproductive age are cancer survivors. They will eventually have been sterilized by their chemotherapy or radiation.

Transplantation of frozen-thawed or fresh ovarian tissue or whole ovary is a delicate procedure aimed to restore fertility to patients who have lost ovarian follicle reserve or have poor quality follicles by delivering a stock of resting non growing follicles that can serve in the future to restore and maintain follicular activity and ovulations that may enable future reproduction. However, high portion of follicles delivered back to the body by transplantation disappear rapidly due to premature follicle activation. The present invention provides pharmaceutical compositions, kits and methods directed to increase graft survival, enable future pregnancy and prolong hormone secretion. Thus, in some embodiments, the pharmaceutical compositions, kits and methods of the invention are directed to subjects undergoing ovarian tissue transplantation or whole ovary transplantation.

Without being bound by any theory or mechanism of action, it is assumed that the process of transplantation is linked to tissue damage such as ischemia, oxidative stress, and fibrosis, all of which have been proposed to induce the follicle activation and loss observed post-transplantation. Accordingly, increasing follicle activation ultimately reduces the reserve of PMFs and thereby reduces the lifespan of the transplanted ovary tissue (graft). Furthermore, transplantation-induced activation as a mechanism of loss is likely the results of the graft preparation and transplantation process, which by necessity isolates the PMFs from the growing (antral, pre-antral and secondary) follicles that maintain the quiescence of the dormant follicles. The absence of growing follicles disrupts the balance between stimulatory and inhibitory follicle activation factors in the graft, thereby instigating follicular activation.

As disclosed herein, unexpectedly rAMH administration during and/or post transplantation restores the normal levels of inhibition, thus preventing the mass follicle activation and loss that would otherwise occur, while preserving the PMF population of the graft. Advantageously, by preventing transplantation-induced follicle activation rAMH improves OTCP-TP clinical outcomes, improves the long term graft endocrine activity and increases fertility potential.

In some embodiments, the present invention provides a method of inhibiting follicle activation in subject undergoing ovary transplantation, the method comprises administering to a subject a therapeutically effective amount of a pharmaceutical composition comprising a compound selected from the group consisting of anti-mullerian hormone, anti-mullerian hormone agonist, and antiMIR of anti-mullerian hormone, following, prior to, or in combination with ovarian tissue or whole ovary transplantation.

The term ‘ovary’ as used herein refers to transplantation of the whole ovary or of parts of the ovary, also termed herein ‘ovarian tissue’. Ovary transplantation usually includes freezing the oocyte or ovary tissue of the treated woman (prior to treatment) or of a suitable donor followed by thawing prior to transplantation. The term “freezing” as used herein includes, but is not limited to, slow freezing, cryopreserving and vitrification.

Ovarian tissue freezing has some benefits over egg freezing. For example, transplanting ovarian tissue not only restores fertility but also restores endocrine function.

In animal studies it was shown that frozen ovarian tissue could be successfully thawed and autotransplanted leading to normal ovarian function and live births. Human live birth from orthotropic transplantation of frozen human ovarian tissue was also achieved (e.g. Meirow et al. N. Engl. J. Med. 2005, 353:318-321).

In some embodiments, ovary transplantation comprises OTCP-TP. In some embodiments, ovary transplantation comprises orthotropic transplantation.

In some embodiments, there is provided a method for ovary transplantation the method comprises (a) preparing donor ovarian cortex; (b) preparing recipient ovarian medulla; (c) attaching the donor cortical tissue to the recipient ovarian medulla; and (d) administering to said recipient a pharmaceutical composition comprising a therapeutically effective amount of AMH.

In some embodiments, said administering comprises administering in parallel to said attaching the donor cortical tissue to the recipient ovarian medulla. In some embodiments, said administering comprises administering at least once after said attaching the donor cortical tissue to the recipient ovarian medulla. In some embodiments, said administering comprises administering in parallel to attaching the donor cortical tissue to the recipient ovarian medulla and at least once thereafter. In some embodiments, said administering at least once after said attaching the donor cortical tissue to the recipient ovarian medulla comprises administering daily, for a plurality of days.

In some embodiments, said pharmaceutical composition is introduced to the patient together with the transplanted tissue/ovary. In some embodiments, said pharmaceutical composition is delivered topically, directly to the ovary.

In some embodiments, said graft (ovarian tissue or whole ovary) is covered with alginate, encapsulated within alginate, or otherwise coated with alginate.

In some embodiments, the ovary transplantation is an autologous transplantation.

In some embodiments, there is provided a method for ovary transplantation the method comprises (a) obtaining ovarian cortex from a subject in need thereof; (b) preparing the ovarian cortex, or fragments thereof for transplantation; (c) preparing the ovarian medulla of said subject for transplantation; (d) attaching the ovarian cortex, or fragments thereof, to the ovarian medulla; and (e) administering a pharmaceutical composition comprising a pharmaceutically effective amount of AMH.

In some embodiments, the method further includes preserving the ovarian cortex post step (b) and prior to step (c).

In some embodiments, the ovarian cortex for transplantation is fresh ovarian cortex.

The term “fresh” as used herein refers to tissue that did not undergo freezing, cryopreservation or any other preservation.

As used herein, the ovarian cortex attached to the ovarian medulla is also termed graft.

In some embodiments, the ovarian cortex, or fragments thereof, being attached to the ovarian medulla according to the method disclosed herein is frozen-thawed ovarian tissue graft.

The term “obtaining ovarian cortex” includes, but is not limited to, any one or more of oophorectomy, unilateral oophorectomy or partial oophorectomy.

In some embodiments, said preserving the ovarian cortex comprises freezing, (e.g. cryopreserving) the ovarian cortex.

In the past, frozen ovary transplanted back to the patient have utilized the slow freeze cryopreservation approach. Currently, vitrification for cryopreservation is primarily used in humans. Vitrification technique usually includes use slices (e.g. 10 mm by 10 mm×1 mm) of cortex tissue from each ovary. Following equilibration of the ovarian tissues in suitable media, the ovarian tissues are placed in a minimum volume of solution (virtually “dry”) onto a thin metal strip and submerged directly into sterile liquid nitrogen, after which the strip is inserted into a container and placed into a liquid nitrogen storage tank. For thawing, the container is removed and the cryotissue metal strip is immersed directly into a solution at 37° C. supplemented with sucrose for a few seconds or minutes. Then, ovary tissues are transferred into a similar solution for a few minutes at room temperature and washed in the solution prior to viability analysis or transplantation.

In some embodiments, preparing the ovarian cortex for transplantation comprises thawing.

Several techniques have been described for transplantation of the ovarian cortex. For example, as exemplified herein, transplantation of the ovarian cortex refers to transplantation of sliced pieces of ovarian cortex. The slices may be a few millimeter long, wide and thick, for example, X mm long, Y mm wide and Z mm think, where any one of X, Y or Z is within the range of 0.1 mm to 15 mm. The ovarian cortical slices can be transplanted under the surface of the cortex in the recipient ovary.

In some embodiments, the subject in need thereof is a subject having a disease or disorder requiring treatment, wherein said obtaining ovarian cortex from a subject in need thereof occurs prior to said treatment.

In some embodiments, said attaching the ovarian cortex, or fragments thereof, to the ovarian medulla occurs post said treatment.

In some embodiments, said treatment is treatment reduces ovarian reserve. In some embodiments, said treatment is treatment that may cause infertility. In some embodiments, said treatment comprises any one or more of chemotherapy and radiation. In some embodiments, said radiation is pelvic irradiation.

Destruction of ovarian follicle reserve is a major side effect of various acute insults, including, but not limited to, chemotherapy. The impact of chemotherapy on fertility is directly dependent on the survival or loss of the dormant oocytes in the primordial follicles that comprise the ovarian follicle reserve. Chemotherapy induces distinct short and long-term effects on the ovary. The immediate effect, occurring during treatment, includes temporary amenorrhea. The greater long-term effect includes damage caused to the primordial follicle pool. Though total loss of the primordial follicle population may occur, resulting in immediate and permanent sterilization, the more common damage is partial loss of the primordial follicle reserve. If sufficient primordial follicles remain, the amenorrhea induced by the loss of the growing follicle population may be short lived. However, the reduction of the primordial follicle pool decreases the remaining window of fertility available to the patient, resulting in permanent amenorrhea and premature menopause.

Most classes of cytotoxic drugs target rapidly dividing cells, interrupting essential cell processes and arresting cellular proliferation. Alkylating agents are not cell-cycle specific and are cytotoxic even when cells are at rest although proliferating cells are known to be more sensitive to their effects. Histological studies on human tissue show that chemotherapy causes a drastic loss of primordial follicle stockpiles. Paclitaxel and cisplatin have been observed to decrease the number of primordial follicles in mice and rats, which may be due to a direct effect of the treatment on follicles or an indirect effect via another cell type such as stroma.

In some embodiments, the subject in need thereof is a healthy woman wishing to preserve her fertility for nonmedical reasons.

Thus, the present invention provides pharmaceutical compositions, kits and use thereof for inhibiting or preventing follicle activation induced by, or during, ovarian transplantation. The compositions and kits of the invention comprise AMH, including AMH agonist or antiMIR of AMH and a combination thereof. The compositions and kits of the invention may be used prior to, after or in combination with the ovarian transplantation.

The terms “follicle activation”, “initiation of follicle growth” and “initial recruitment of follicle” as used herein are interchangeable and generally refer to the transition of dormant/primordial follicles into growing follicles.

The terms “premature follicle activation”, “early follicle activation” and “follicle burn out” a used herein are interchangeable and refer to processes induced by acute insults which may eventually cause, or result in, loss of fertility. The aforementioned processes may results with menopause earlier than expected.

Anti-mullerian hormone, also termed hereinafter “AMH”, typically refers to a protein designated by NCBI Accession No.: P03971. It has been also termed Müllerian inhibiting factor (MIF), Müllerian-inhibiting hormone (MIH), and Müllerian-inhibiting substance. AMH as used herein encompasses the full AMH sequence, homologs, analogs, variants and derivative of the AMH protein or a fragment thereof, with the stipulation that the AMH activity is preserved, including, but not limited to, recombinant AMH (rAMH), recombinant human AMH (rhAMH), and C-terminal fragment of rhAMH. A mathematical model simulating the female reproductive cycle, predicted that AMH could be used to delay, naturally, menopause (Margolskee et al., J. Theor. Biol., 326:21-35, February 2013).

Without being bound by any theory or mechanism, AMH inhibits or prevents follicle activation by inhibiting or preventing recruitment of primordial follicles into the pool of growing follicles, thereby preventing undesired acceleration effect on growing follicle resulting in follicle exhaustion, as for example induced by a disease, a syndrome, invasive procedures and/or medicaments, such as, chemotherapy and ovarian transplantation. Under normal physiological conditions, AMH protects the reserve of primordial follicles. Under acute insult the follicle reserve may undergo apoptosis and/or may be reduced for lack of nutrients due to destructions of the vascular system that nourishes the follicles. Unexpectedly, as shown herein, AMH protects the PMF reserve in treated grafts. This effect has been demonstrated herein by significant increase in PMF numbers and reduced granulosa cell proliferation (Ki67 staining) compared to untreated grafts, as well as a reduction in the ratio of GF to PMF. Negative correlation between the presence of proliferation marker Ki67 and AMH in the graft tissue demonstrated that AMH directly prevents transplantation-induced activation of the PMF population, thereby preserving the PMF population of the graft. Thus, use of AMH or its derivatives may be carried out before, after or in combination with ovary transplantation.

The term “agonist” as used herein refers to any chemical substance, a fragment of AMH protein, a derivative of AMH or a modified AMH protein, which capable of activating the AMH receptor, resulting with the inhibition of follicle activation induced by an acute insult.

As used herein and further detailed below, the term “inhibiting follicle activation” or “preventing follicle activation” refers to a transient or permanent condition wherein some or all follicles are maintained in their primordial stage.

The term “antiMIR” refers to contiguous nucleic acids, DNA or RNA, which are complementary to micro-RNA or miRNA. The antiMIR binds to the miRNA and inhibits the silencing/degrading activity it has upon the mRNA of a target gene. This results in elevation of the target gene expression. The antiMIR of the invention is targeted for miRNA that silence the AMH gene. In some embodiments, the present invention provides a method of preventing artificially induced follicle activation, comprising the step of administering antiMIR of AMH.

The term “antiMIR of AMH” refers to a molecule that inhibits AMH silencing by miRNA.

The term “complementary” in the context of the present invention refers to antiMIR sequence that has at least 90%, 95%, or 100% identity to a complementary sequence of miRNA of AMH.

The term “inhibiting” as used herein includes, but is not limited to, preventing, attenuating, impeding, reducing to a certain extent, complete inhibition and/or partial inhibition.

As used herein, the term “therapeutically effective amount” refers to an amount of a formulation or composition which is effective to inhibit or prevent, at least partially, follicle activation in a living organism to whom it is administered over some period of time.

The present invention further provides a method for inhibiting or preventing the induced follicle activation in a subject in need thereof by increasing the activity of the AMH receptor. Increasing the activity of an AMH receptor may be obtained, for example, by elevating the AMH or AMH agonist amounts. Administering AMH per se is one approach for elevating AMH amount. Another approach is by inducing overexpression of a gene encoding for AMH. Overexpression of AMH could be achieved by gene therapy mediated by adenovirus and lentivirus vectors.

The AMH protein hormone may be isolated and purified by methods selected based on properties revealed by its sequence. Purification can be achieved by protein purification procedures such as chromatography methods (gel-filtration, ion-exchange and immunoaffinity), by high-performance liquid chromatography (HPLC, RP-HPLC, ion-exchange HPLC, size-exclusion HPLC, high-performance chromatofocusing and hydrophobic interaction chromatography) or by precipitation (immunoprecipitation). Polyacrylamide gel electrophoresis can also be used to isolate the AMH protein based on the molecular weight of the protein, charge properties and hydrophobicity. For example, Picard et al. describes an improved method for the purification of anti-Müllerian hormone from incubation medium of bovine fetal testes (Mol Cell Endocrinol., 1984, 34(1):23-29).

According to alternative embodiments, AMH or its equivalents may be produced by the use of recombinant DNA techniques as are well known to one skilled in the art. Nucleic acid sequences which encode for the proteins of the invention may be incorporated in a known manner into appropriate expression vectors (i.e. recombinant expression vectors). Possible expression vectors include, but are not limited to, cosmids, plasmids, or modified viruses (e.g. replication defective retroviruses, adenoviruses and adeno-associated viruses, lentiviruses, herpes viruses, poxviruses), so long as the vector is compatible with the host cell used. The expression “vector . . . compatible with the host cell” is defined as contemplating that the expression vector(s) contain a nucleic acid molecule of the invention and attendant regulatory sequence(s) selected on the basis of the host cell(s) to be used for expression, said regulatory sequence(s) being operatively linked to the nucleic acid molecule. “Operatively linked” is intended to mean that the nucleic acid is linked to regulatory sequence(s) in a manner which allows expression of the nucleic acid. Suitable regulatory sequences may be derived from a variety of sources, including bacteria, fungal, or viral genes (for example, see the regulatory sequences described in Goeddel, Gene Expression Technology: Methods in Enzymology 185, Academic Press, San Diego, Calif. (1990)). Selection of appropriate regulatory sequence(s) is dependent on the host cell(s) chosen, and may be readily accomplished by one of ordinary skill in the art. Examples of such regulatory sequences include the following: a transcriptional promoter and enhancer, RNA polymerase binding sequence, or a ribosomal binding sequence (including a translation initiation signal). Depending on the host cell chosen and the expression vector employed, other additional sequences (such as an origin of replication, additional DNA restriction sites, enhancers, and sequences conferring inducibility of transcription) may be incorporated into the expression vector.

It is to be understood that the pharmaceutical compositions, kits and methods of the invention are directed for treating women. Thus, the terms ‘subject’ and ‘subject in need thereof’ refer to females, commonly, in their reproductive, fertile, years, including, women and adolescent children. The term ‘reproductive years’ refers in general to the age following puberty and prior to menopause.

In some embodiment, the subject is a subject having a disease or disorder associated with loss of follicle activation and/or loss or reduced fertility.

In some embodiment, the subject is a subject having a disease or disorder associated with loss of follicle activation and/or loss or reduced fertility which are induced by a medical treatment.

In some embodiments, the subject is undergoing treatment with an agent that induces follicle loss. In some embodiments, said agent is a chemotherapeutic agent.

In some embodiments, the subject is a subject having cancer. In some embodiments, the subject having cancer is being treated with anti-cancer therapy prior to and/or in parallel to treatment for inhibiting follicle activation. In some embodiments, the anti-cancer therapy is chemotherapy.

In some embodiments, the subject is a healthy subject wishing to undergo ovary transplantation for non-medical reasons, or for improving fertility.

The term “healthy subject” as used herein refers to subject that is not in need of chemotherapy or treatment that induces or enhances follicle burn out.

In other embodiments, the anti-cancer therapy is radiotherapy. In some embodiments, the anti-cancer treatment results in accelerated or premature follicle activation.

The term “cancer” is used herein in its broadest sense and refers to a family of diseases characterized by uncontrolled cell growth. It includes, but is not limited to, adrenocortical carcinoma, anal cancer, bladder cancer, brain stem glioma, brain tumor, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, endometrial cancer, esophageal cancer, extrahepatic bile duct cancer, ewings family of tumors, extracranial germ cell tumor, eye cancer, intraocular melanoma, gallbladder cancer, gastric cancer, germ cell tumor, extragonadal germ cell tumor, gestational trophoblastic tumor, head and neck cancer, hypopharyngeal cancer, islet cell carcinoma, laryngeal cancer, acute lymphoblastic leukemia, oral cavity cancer, liver cancer, lung cancer, small cell lymphoma, AIDS-related lymphoma, central nervous system (primary) lymphoma, cutaneous T-cell lymphoma, hodgkin's disease, non-hodgkin's disease, malignant mesothelioma, melanoma, merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian epithelial cancer, ovarian germ cell tumor, ovarian low malignant potential tumor, pancreatic cancer, exocrine pancreatic cancer, paranasal sinus and nasal cavity cancer, parathyroid cancer, pheochromocytoma cancer, pituitary cancer, plasma cell neoplasm, rhabdomyosarcoma, rectal cancer, renal cell cancer, salivary gland cancer, sezary syndrome, kaposi's sarcoma, skin cancer, small intestine cancer, soft tissue sarcoma, thymoma, malignant, thyroid cancer, urethral cancer, uterine cancer, sarcoma, unusual cancer of childhood, vaginal cancer, vulvar cancer, or wilms' tumor.

In some embodiments, the subject in need is having an accelerated follicle to activation disease or disorder. In some embodiments, the disease is a genetic disorder such as Turner syndrome. In other embodiments, the disease is Galactosemia. In other embodiments, the disease is endometriosis.

Turner syndrome refers to a chromosomal condition that affects development in females. Turner syndrome occurs when one normal X chromosome is present in a female's cells and the other sex chromosome is missing or structurally altered. Turner syndrome is characterized by an early loss of ovarian function and accelerated follicle activation may be one of the causes for this phenomenon.

Galactosemia is an inherited disorder characterized by inability to metabolize the sugar galactose properly. One of the symptoms of Galactosemia is accelerated follicle activation.

In some embodiments, the present invention provides a pharmaceutical composition comprising a compound selected from the group consisting of anti-mullerian hormone, anti-mullerian hormone agonist, and antiMIR of anti-mullerian hormone, for use in inhibiting follicle activation in subject in need thereof.

In some embodiments, the pharmaceutical composition further comprises a pharmaceutically acceptable carrier, excipient or diluent.

As used herein, a “pharmaceutical composition” refers to a preparation of one or more of the active ingredients described herein, for example, AMH molecule, AMH agonist, antiMIR of AMH, with non-active (inert) components, such as, physiologically suitable carriers and excipients. The purpose of a pharmaceutical composition is to facilitate administration of a compound to a subject.

Herein, the phrases “therapeutically acceptable carrier”, “physiologically suitable carriers and excipients” and “pharmaceutically acceptable carrier”, which may be used interchangeably, and refer to a carrier or a diluent that does not cause significant irritation to an organism and does not abrogate the biological activity and properties of the administered compound.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

As used herein, a “carrier” refers to any substance suitable as a vehicle for delivering an amino acid or a nucleic acid molecule of the present invention to a suitable in vivo or in vitro site. As such, carriers can act as a pharmaceutically acceptable excipient of a therapeutic composition containing a molecule of the present invention. Carriers of the present invention include: (1) excipients or formularies that transport, but do not specifically target a nucleic acid molecule to a cell (referred to herein as non-targeting carriers); and (2) excipients or formularies that deliver an amino acid or nucleic acid molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- and o-cresol, formalin and benzol alcohol. Therapeutic compositions of the present invention can be sterilized by conventional methods.

The pharmaceutical compositions of the present invention may be manufactured by processes well known in the art for the preparation of pharmaceutically acceptable compositions intended for administration to a subject, e.g. by means of conventional mixing, dissolving, granulating, grinding, pulverizing, dragee-making, levigating, emulsifying, encapsulating, entrapping or lyophilizing processes.

The compositions described herein may be prepared such that an effective quantity of the active substance (e.g. AMH) is combined in a mixture with a suitable pharmaceutically acceptable vehicle as known in the art. On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids.

Furthermore, the pharmaceutical compositions according to the invention may to comprise one or more stabilizers, such as, for example, carbohydrates including sorbitol, mannitol, starch, sucrose, dextrin and glucose, proteins such as albumin or casein, and buffers like alkaline phosphates.

In some embodiments, administering the pharmaceutical composition comprises administering via a route selected from the group consisting of: subcutaneous, topical, transdermal, oral, buccal, sublingual, sublabial, intradermal, intravaginal or combinations thereof. Each possibility is a separate embodiment of the invention.

In some embodiments, administering the pharmaceutical composition comprises direct delivery to the ovary. In some embodiments, administering the pharmaceutical composition comprises direct injection to the ovary. In some embodiments, administering the pharmaceutical composition comprises systemic administration.

In some embodiments, the pharmaceutical composition is administered by direct delivery to the ovary. In some embodiments, the pharmaceutical composition is delivered to each ovary. In some embodiments, the pharmaceutical composition is delivered to each ovary prior to initiation of the acute insult.

Administration of an “effective amount” of the pharmaceutical compositions of the present invention refers to administration of an amount effective at dosages and for periods of time, necessary to elicit a desired therapeutic response in a human. A therapeutically effective amount of a substance may vary according to the follicle activator factor or cause, age, sex, and weight of the recipient. Dosage regimes may be adjusted to provide the optimum therapeutic response. For example, several divided doses may be administered daily or on at periodic intervals, and/or the dose may be proportionally reduced as indicated by the exigencies of the therapeutic situation. The amount of pharmaceutical composition for administration will depend on the route of administration, time of administration and varied in accordance with individual subject responses.

There is provided, in some embodiments, a kit for the inhibiting or preventing follicle activation in a subject in need thereof, the kit comprising:

(i) a first packaging containing a pharmaceutical composition comprising a compound selected from the group consisting of anti-mullerian hormone, anti-mullerian hormone agonist, and antiMIR of anti-mullerian hormone; and

(ii) written instructions for of use of said pharmaceutical composition for inhibiting follicle activation in said subject.

In some embodiments, the kit further comprises a second packaging containing a pharmaceutical composition comprising at least one anti-cancer agent.

In some embodiments, the kit further comprises a third packaging containing a pharmaceutical composition comprising at least one follicle reserve protective compound and a pharmaceutically acceptable carrier, diluent or excipient.

In some embodiments, the at least one follicle reserve protective compound comprises sphingosine-1-phosphate, Tamoxifen, GnRH, trichloro(dioxoethylene-O,O′) or a combination thereof.

In some embodiments, the pharmaceutical composition in the first packaging may further comprise at least one anticancer agent.

The term “kit” as used herein is interchangeable with the term package, and refers to packages of pharmaceutical formulations containing any one or more of anti-mullerian hormone, anti-mullerian hormone agonist, antiMIR of anti-mullerian hormone and further containing, together, or in a different packaging, the anticancer agent and/or at least one follicle reserve protective compound. Accordingly, the kit may be organized to indicate a single formulation or combination of formulations to be taken at each desired treatment regimen as specified in written instructions encompassed in the kit.

The kit may optionally contain instructions for administering the pharmaceutical composition to a subject having a disease associated with premature follicle activation or having a condition requiring to inhibit or attenuate follicle activation in order to protect fertility.

In some embodiments, the kit contains packaging or a container with each of said first and second and third pharmaceutical compositions, formulated for the desired delivery route. Suitably, the kit contains instructions on dosing and an insert regarding the active agent. Optionally, the kit may further contain instructions for monitoring circulating levels of product(s) and material(s) that may be used for evaluating treatment efficacy. For performing such evaluation assays that kit may further include reagents, well plates, containers, markers or labels, and the like. Such kits are readily packaged in a manner suitable for treatment of a desired indication. The kit may also contain instructions for use of a delivery device. Other suitable components to include in such kits will be readily apparent to one of skill in the art, taking into consideration the desired indication and the delivery route.

The compositions described herein can be a single dose or for continuous or periodic discontinuous administration. For continuous administration, the package or kit may include each of the pharmaceutical compositions in their dosage unit (e.g., solution, lotion, tablet, pill, or other unit described above or utilized in drug delivery), and optionally instructions for administering the doses daily, weekly, or monthly, for a predetermined length of time or as prescribed. When the pharmaceutical compositions are to be delivered periodically in a discontinuous fashion, the package or kit may include placebos during periods when the pharmaceutical compositions are not delivered. When varying concentrations of a composition, of the components of the composition, or the relative ratios of the components of the pharmaceutical composition or the ratio of the first pharmaceutical composition to the second pharmaceutical composition over time is desired, the package or kit may contain a sequence of dosage units which provide the desired variability.

A number of packages or kits are known in the art for dispensing pharmaceutical agents for periodic oral use. In some embodiments, the package has indicators for each period. In other embodiments, the package is a labeled blister package, dial dispenser package, or bottle.

The packaging means of a kit may itself be geared for administration, such as an inhaler, syringe, pipette, eye dropper, or other such apparatus, from which the pharmaceutical composition(s) may be applied to an affected area of the body, such as the arms, injected into a subject, or even applied to and mixed with the other components of the kit.

The compositions of the kit of the invention also may be provided in dried or lyophilized forms. When reagents or components are provided as a dried form, reconstitution generally is by the addition of a suitable solvent. It is envisioned that the solvent also may be provided in another packaging of the kit.

The kit of the present invention also will typically include a means for containing the vials in close confinement for commercial sale such as, e.g., injection or blow-molded plastic containers into which the desired vials are retained. Irrespective of the number or type of packages and as discussed above, the kit also may include, or be packaged with a separate instrument for assisting with the injection/administration or placement of the composition within the body. Such an instrument may be syringe, pipette, forceps, measuring spoon or any such medically approved delivery means.

In some embodiments, the pharmaceutical compositions of the kit are provided in the presence or absence of one or more of the carriers or excipients described above.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1: Ovarian Cortical Tissue Preparation, Transplantation and Analysis

Five healthy adult female marmoset monkeys (Callithrix jacchus) at the age range of 13 to 23 months were made available for ovaries excision. Fresh bovine ovaries were collected from lactating milking cows, ages 1.5-3 years (n=12). Ovarian human tissues were harvested from eight donors (age range 13-26 years).

Cortical tissue strips were obtained from marmoset (n=15), bovine (n=15) and human (n=46). The cortical tissue was obtained by removal of the underlying stromal tissue. Slow freezing cryopreservation was performed according to previously published protocols. Briefly, tissue fragments were equilibrated with 0.1M sucrose and 1.5M DMSO for 30 minutes following introduction into the cryopreservation device. A multi-gradient slow freezing protocol cooled the samples to −150° C. following by storage in liquid nitrogen (LN).

Prior to transplantation, tissues were thawed rapidly by equilibrating the samples in decreasing DMSO gradient solutions in 0.1M sucrose and cut into 2×3×2 mm pieces (Marmoset, n=29: n=15 for transplantation experiment and n=14 for transplantation+AMH experiment; Bovine, n=37; Human, n=46). Fresh thawed cortex from each experiment was embedded in 4% paraffin and further studied.

Seven-weeks-old castrated male immunodeficient mice (n=60) were used as recipient animals. Mice were castrated at 4 weeks and maintained at 28° C. under controlled SPF conditions. Xenotransplantation experiments commenced at least 2-weeks post-orchidectomy.

Ovarian tissue transplantation surgical procedures were performed as described previously. Briefly, a longitudinal incision was made in the dorsum of the anesthetized recipient mouse, the skin was lifted and the ovarian cortical strips were inserted subcutaneously. Size of transplanted tissue from all species was 2×3×2 mm. Each graft was attached in place with a 6-0 non-absorbable prolene suture. Transplanted tissues were recovered after three (3) or seven (7) days.

In a separate experiment, frozen marmoset cortical tissues were thawed, 2×3×2 mm cortical strips were prepared (n=14) and transplanted into castrated SCID mice as described above. rAMH, diluted in PBS, was injected (10 μg in 200 μl, per mouse, i.p.) at the time of transplantation and once daily for 3 consecutive days at 10 μg per mouse, while control mice were injected with PBS. Daily injection protocol was determined according to preliminary experiments using labeled rAMH and rAMH dosage was initially determined according to previous studies.

As the first set of experiments indicated maximal follicle activation and PMF loss in all species (in the absence of AMH) at three days post transplantation, the animals were sacrificed three days post transplantation. The grafts were removed and processed for histological follicle counting analysis and immunohistochemistry.

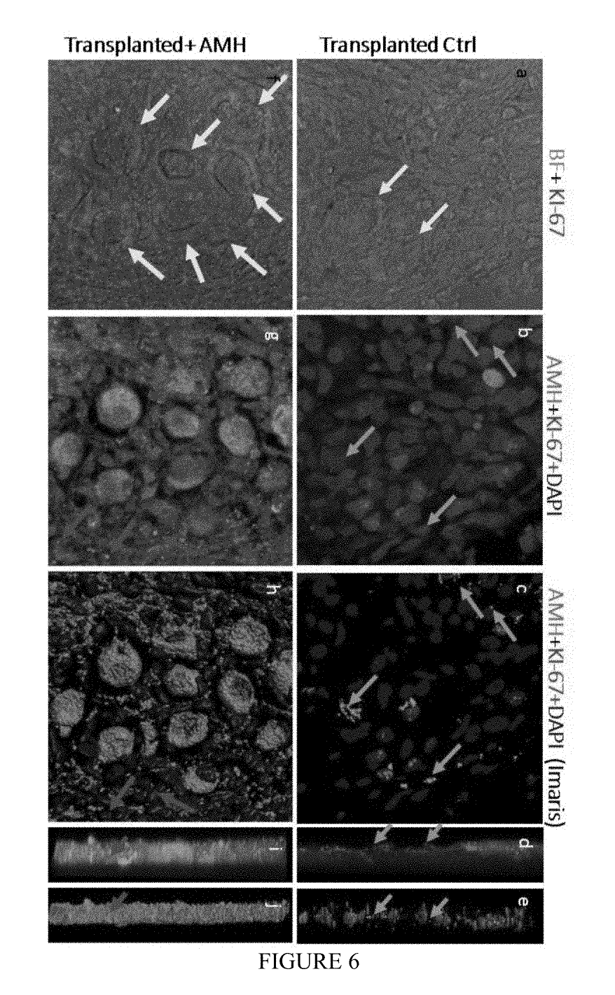

Recovered grafts and control frozen thawed non transplanted samples were fixed in 4% paraformaldehyde, embedded in paraffin and serial 5 μm sections were prepared from the whole grafts for follicle counts and immunohistochemistry. Follicle classification as primordial or growing was performed on every sixth section of Hematoxylin and Eosin stained sections by two independent observers under a light-microscope (slides/graft: 8 for Marmoset, 9 for Bovine, 6 for Human). Only non-atretic follicles with clearly visible oocyte circled by granulosa-cell layers were referred to in results since no significant difference was found in the number of atretic follicles among the groups. PMF were defined as an oocyte surrounded by a single layer of flattened pre-granulosa cells. GF include follicles in all developing stages. In Sirius red collagen staining, collagen fibers stained (FIGS. 6D and 6E) in a counterstain compared to the tissue (FIGS. 6I and 6J).

For immunohistochemistry, sections were incubated with primary and then secondary antibodies: Ki-67, GDF9 or AMH.

For immunofluorescence, Donkey anti-goat IgG antibodies with Cy2 dye or Cy3 dye were used as secondary antibodies for AMH or Ki-67 respectively. Murine LLC tumor was used as positive controls for Ki-67 immunostaining, 6 weeks old murine ovary was used as positive control for GDF-9 and AMH immunostainings. Ovary tissue from each species exposed to secondary antibody only was used as negative control.

### Example 2: Follicle Loss Post Transplantation

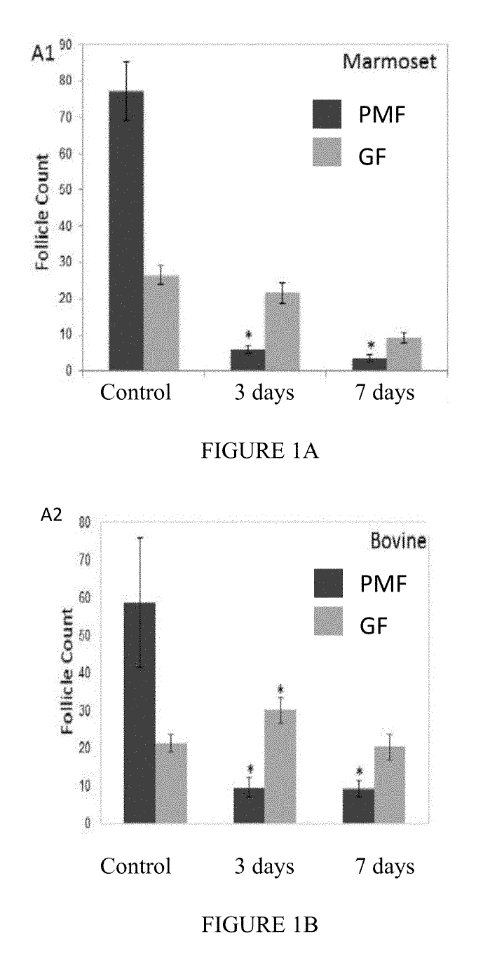

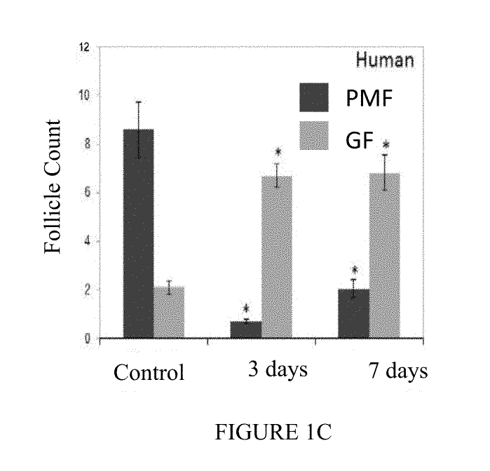

All grafts were recovered and evaluated. Grafts of all transplanted tissue species demonstrated significant and dramatic primordial follicle (PMF) loss already three days post transplantation compared to untransplanted controls (FIGS. 1A-1C, black columns), as follows: 92% (5.72±0.97 vs. 76.80±6.91) marmoset grafts; 83% (9.58±1.01 vs 58.67±6.70) bovine grafts; and 91% (0.70±0.12 vs 8.60±1.44) in human grafts. The results were significant (FIG. 1A-1C: *=p<0.001). In all species, no additional significant PMF loss was observed in 7d grafts compared to 3d grafts.

In contrary to the massive drop in PMF population (FIGS. 1A-1C, black columns), growing follicles (GF) numbers were high post transplantation (FIGS. 1A-1C, gray columns), reaching maximal numbers at three days compared with untransplanted control and post transplantation 7 days evaluation (bovine, human; FIGS. 1B-1C).

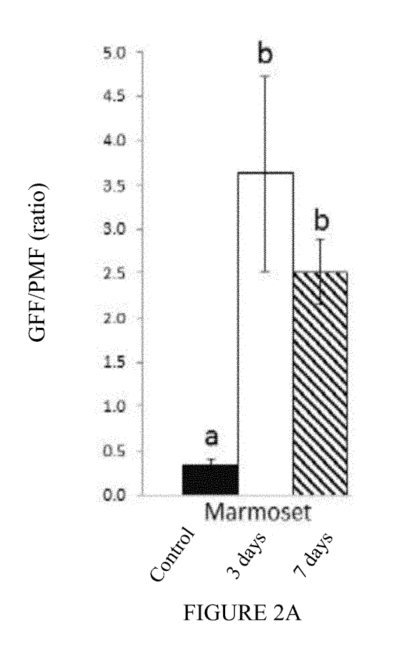

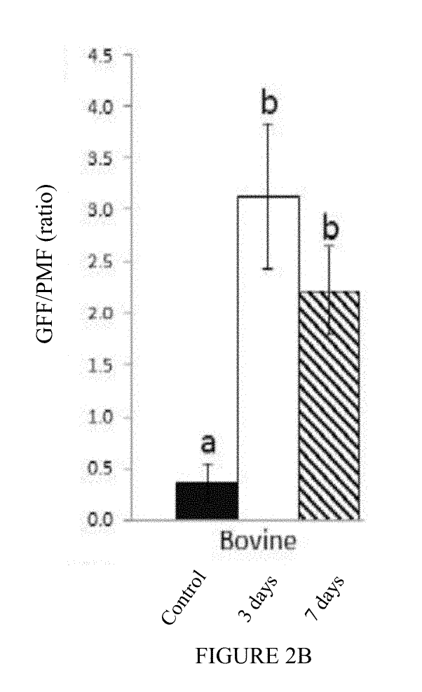

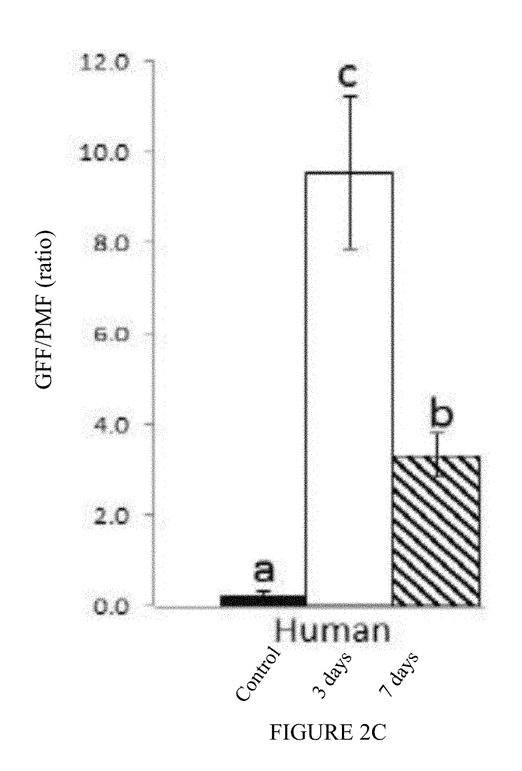

The relative distribution in follicle subclass was expressed by calculating the ratio of growing to primordial follicles (GF/PMF), where changes in this ratio post transplantation provide a measure of follicle activation (Kalich-Philosoph et al., Sci. Transl. Med., 5: 185ra162, 2013). Untransplanted control tissues from all species exhibited a similar GF/PMF ratio of approximately 0.3, indicating that PMF comprised the substantial majority of the follicle population. However, 3 and 7 days post transplantation the GF/PMF ratios in all species increased significantly showing maximal ratio at three days post transplantation (p<0.02; FIGS. 2A-2C, where ‘a’ and ‘b’ represents H&E and Ki-67 staining in untransplanted control, respectively).

Results were expressed as mean follicle count per section±SE or GF/PMF ratio±SE. Data were subjected to one-way analysis of variance (ANOVA) and all-pairs Tukey-Kramer HSD test. P-values lower than 0.05 were considered statistically significant.

### Example 3: Increased Proliferation and Fibrosis Observed in Transplanted Grafts

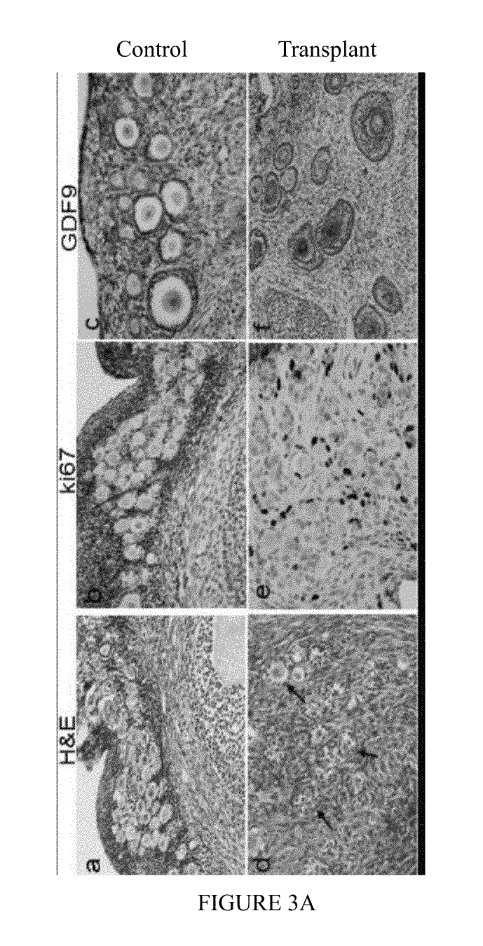

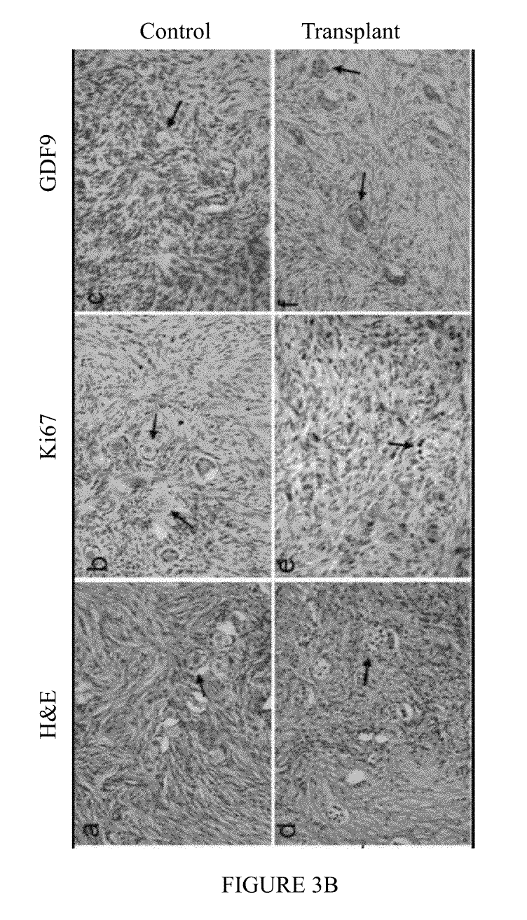

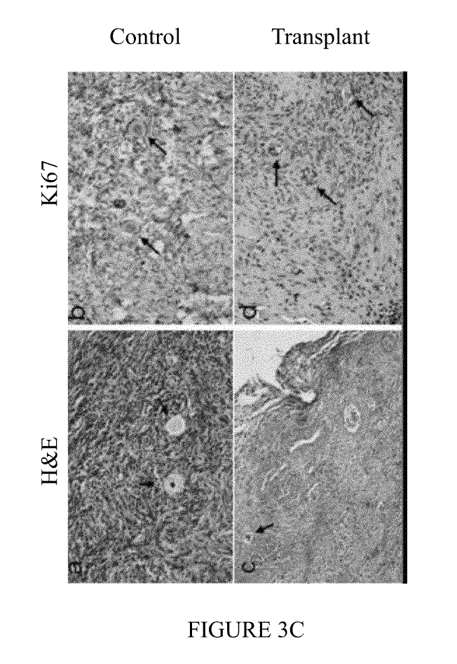

Seven days from grafting, stromal changes, specifically fibrosis, were observed in all grafted samples compared to untransplanted control (FIGS. 3A-3B, panels d vs. panels a, FIG. 3C, panel c vs. panel a). Increased staining for Ki67 in granulosa cells was observed after seven days in all transplanted grafts, demonstrating proliferation in activated PMF and GF (FIGS. 3A-3B, panels e vs panels b, FIG. 3C, panel d vs panel b). Extensive follicle staining for GDF-9 representing progression from primordial follicle was observed in follicles in grafts of marmoset and bovine (FIGS. 3A-3B, panels f vs panels c).

### Example 4: rAMH Protects Against PMF Activation and Loss in OTCP-TP Model

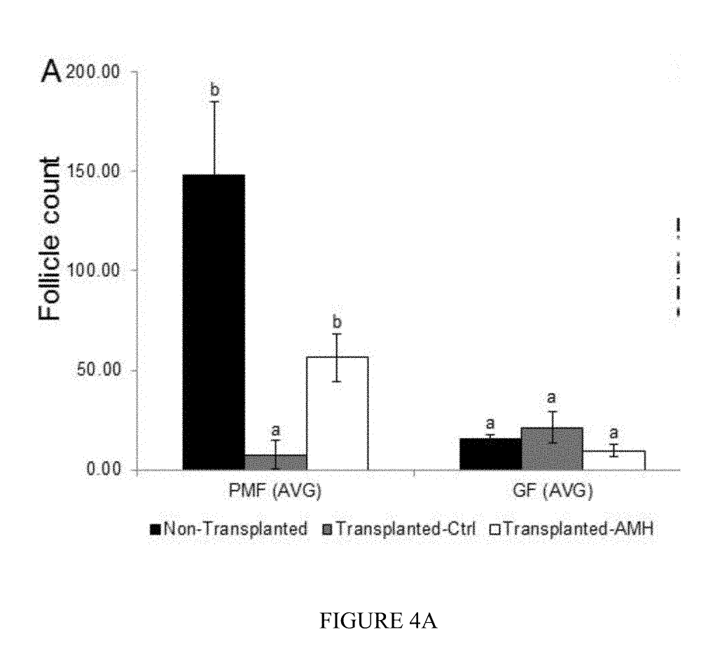

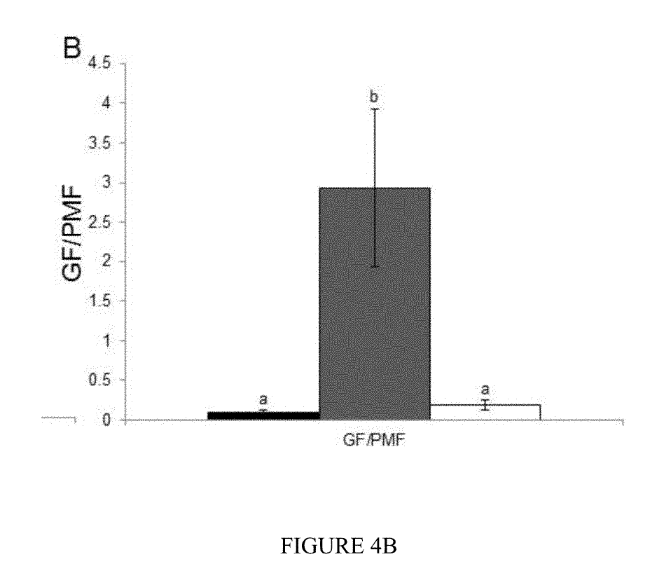

In this set of experiments transplanted marmoset grafts (FIG. 4A, gray columns) also exhibited a significant reduction in the overall follicle numbers (PMF+GF: 82%) three days post transplantation compared to untransplanted control (p<0.014; FIG. 4A, black columns). This reduction was mostly due to decline in PMF numbers (94.81%).

However, rAMH administration protected against PMF follicle loss (FIG. 4A white columns), with 86.40% additionally saved PMF compared to transplanted control (PMF: 56.33±12.12, +rAMH; 7.66±6.99, transplanted control; 148±37.06, untransplanted control; p<0.001). This observation was supported by GF/PMF ratio—which significantly increased in transplanted control (FIG. 4B, gray column) and was normalized in transplanted+ rAMH group (FIG. 4B, white column), compared to untransplanted control (FIG. 4B, black column).

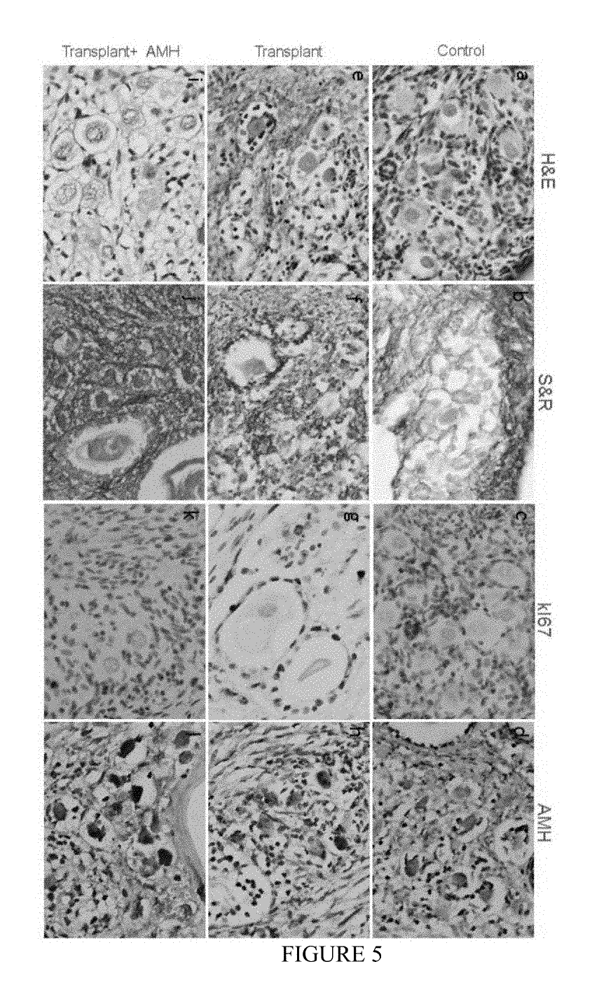

Reconstruction of AMH and Ki-67 protein expression from Z-stack images of mouse ovary was performed, demonstrating reduction in proliferation following rAMH administration, as shown in FIG. 5: transplanted ovaries without (FIGS. 5A-5E: B-fluorescence, C-Imaris, side-view: D-fluorescence, E-Imaris) or with rAMH administration (FIGS. 5F-5J: G-fluorescence, H-Imaris, side-view: I-fluorescence, J-Imaris) were double immunostained with AMH (green) and Ki-67 (red) antibodies. Bright Field (BF)+ red filters showing PMF, tissue morphology and KI-67 signal (FIG. 6A-transplanted control, FIG. 6F-transplanted+ AMH, PMF-black arrows). White arrows mark AMH signal, gray arrows marks Ki-67 signal. Twelve (12) Z-stack images were taken, and Imaris software was used for the three-dimensional reconstruction. Major reduction in Ki-67 expressing proliferating cells was observed in presence of AMH (FIGS. 6G-6J compared with FIGS. 6B-E). The figure clearly show that there was a negative correlation between areas of AMH staining and areas with Ki-67 staining—There is very little Ki-67 staining in areas strongly stained for AMH.

Areas containing high AMH levels were accompanied by reduced Ki-67 levels, in line with AMH inhibition of follicle activation. Grafts from both transplanted groups exhibited a marked increase in collagen expression three days post transplantation compared with untransplanted control (FIG. 5, panels F and J vs 4B). rAMH administration however, had no effect on collagen levels.

### Example 5: AMH Prevents Follicle Loss in Ovaries Treated with Chemotherapy

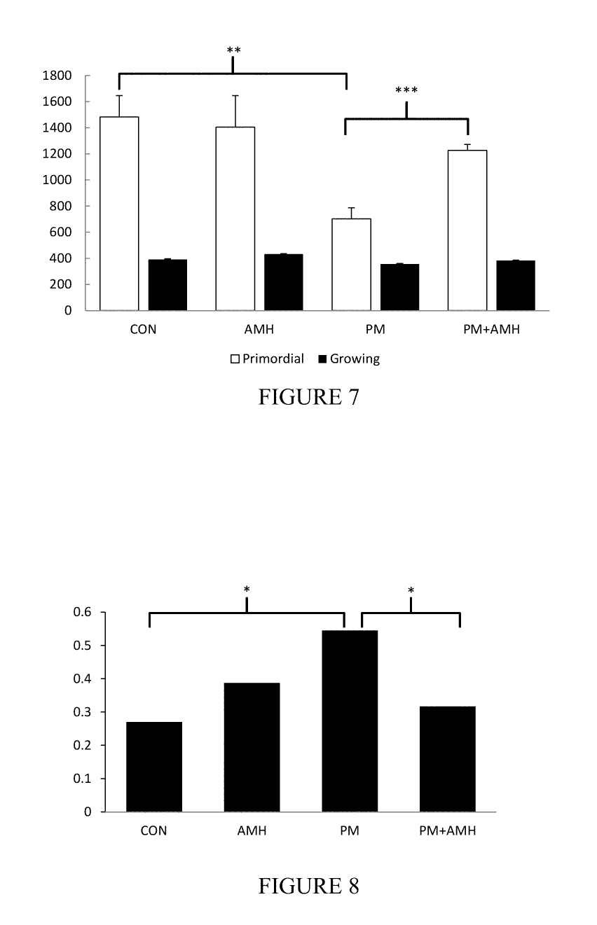

In mice, normal ovarian follicle dynamics are only fully established by approximately day 10 after birth and therefore ovaries from 12-day-old neonatal mice were used to examine the effect of AMH. The ovaries were cultured in-vitro in the presence of the cyclophosphamide metabolite, phosphoramide mustard (PM) for 4 hours, either with or without 200 ng/ml AMH (PM+AMH or PM, respectively) and then washed and continued culture was also with or without AMH. Control ovaries were cultured with medium alone (No Rx) or with only AMH (AMH). Ovaries were removed after day 4 and day 7 in culture and processed for histological analysis. The number of primordial and growing follicles was counted. No difference in primordial or growing follicle numbers was observed between ovaries cultured in regular media alone and ovaries cultured with media and AMH (FIG. 7). A significantly reduced numbers of primordial follicles was observed in ovaries exposed to PM alone compared with untreated ovaries (FIG. 7, white columns PM vs. Control; p<0.01). However, in ovaries exposed to PM together with AMH a significantly greater numbers of primordial follicles was observed relative to PM alone (FIG. 7, white columns PM+AMH vs. PM; p<0.001).

Ovaries from 12-day-old neonatal mice were further incubated with a different chemotherapy agent-cisplatin, with or without AMH. Control ovaries were cultured with medium alone or with AMH. Ovaries were removed after day 4 and day 7 in culture and processed for histological analysis. The number of primordial and growing follicles was counted. Primordial follicle count in the presence of AMH and cisplatin is higher than in the presence of cisplatin alone (results not shown).

Thus, treatment with AMH is suitable for fertile women undergoing chemotherapy as it offers an advantageous platform for preserving their fertility and prolong maintenance of the ovarian function due to larger follicle stockpile which survive treatment.

It is worth noting that in order to obtain valuable data, studies related to primordial follicles should be conducted in primordial follicles from ovaries that completed their perinatal packaging, namely, ovaries from mice that are at least 10 days old since normal ovarian follicle dynamics only begin after about 10 days. At that stage the balanced dynamic of follicle activation/suppression is stabilized. Ovaries at an earlier stage (such as ovaries of 2-day-old mice) have not completed their perinatal packaging, nor reached normal ovarian follicle dynamics, leaving many ‘naked oocytes’.

### Example 6: AMH Improves Growing:Dormant Follicles Ratio Under Chemotherapy

The ratio of growing/dormant follicles was examined in ovaries treated with the chemotherapy drug with and without AMH (FIG. 8). Significant differences between the treatments were observed on day 4 and 7. The ratio of growing to dormant follicles was greatest in the ovaries exposed to PM alone. This ratio was significantly improved in ovaries exposed to PM+AMH (p<0.05).

Overall, the results indicate that AMH reduces chemo-induced follicle activation, suggesting its potential in protecting follicle reserve in young female cancer patients.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method of inhibiting premature follicle activation comprising administering to a subject in need thereof a therapeutically effective amount of a pharmaceutical composition comprising a compound selected from the group consisting of anti-mullerian hormone, anti-mullerian hormone agonist, and antiMIR of anti-mullerian hormone, wherein the premature follicle activation is induced by ovarian transplantation.

2. The method of claim 1, wherein the compound is anti-mullerian hormone.

3. The method of claim 1, wherein the ovarian transplantation comprises transplantation of ovarian tissue or whole ovary.

4. The method of claim 3, wherein said pharmaceutical composition is administered during any one or more of the following: prior to said transplantation, during said transplantation, and post said transplantation.

5. The method of claim 3, wherein said pharmaceutical composition is administered prior to and during, said transplantation.

6. The method of claim 3, wherein said pharmaceutical composition is administered simultaneously with said transplantation.

7. The method of claim 6, wherein said pharmaceutical composition is further administered after said transplantation.

8. The method of claim 1, further comprising administering to said subject at least one follicle reserve protective compound.

9. The method of claim 1, wherein said subject is a female subject in her reproductive years.

10. The method of claim 1, wherein said transplantation comprises ovarian tissue cryopreservation and transplantation.

11. The method of claim 1, wherein said transplantation is an autologous transplantation.

12. The method of claim 1, wherein said transplantation is an orthotropic transplantation.

13. A method for ovarian tissue transplantation, the method comprising:
a. obtaining ovarian cortex from a first subject;
b. preparing the ovarian cortex, or fragments thereof for transplantation;
c. preparing an ovarian medulla of a second subject for transplantation;
d. attaching the ovarian cortex prepared in step (b), or fragments thereof, to the ovarian medulla prepared in step (c); and
e. administering a pharmaceutical composition comprising a pharmaceutically effective amount of AMH.

14. The method of claim 13, wherein said first subject and said second subject are the same subject.

15. The method of claim 13, wherein said first subject is a donor and said second subject is a recipient.

16. The method of claim 13, further comprising preserving the ovarian cortex, or fragments thereof prior to step (b).

17. The method of claim 16, wherein preserving comprises freezing.

18. The method of claim 16, wherein preparing comprises thawing.

19. The method of claim 13, wherein the subject is having a disease or disorder requiring treatment, and wherein said obtaining ovarian cortex is performed prior to said treatment.

20. The method of claim 13, wherein said subject is a healthy woman wishing to preserve her fertility for nonmedical reasons.
